(19) United States
(12) Patent Application Publication
(10) Pub. No.: US 2024/0028916 A1
(43) Pub. Date: Jan. 25, 2024

(54) Method And Device For Drug Combination Design By High-Throughput Platform And Machine Learning For Optimizing

(71) Applicant: University of Science & Technology Beijing, Beijing (CN)

(72) Inventors: Dawei ZHANG, Beijing (CN); Jingzhi YANG, Beijing (CN); Lingwei MA, Beijing (CN); Xiangping HAO, Beijing (CN); Hongchang QIAN, Beijing (CN)

(21) Appl. No.: 17/978,472

(22) Filed: Nov. 1, 2022

(30) Foreign Application Priority Data
Jul. 19, 2022 (CN) 202210845111.7

(51) Int. Cl. G06N 5/02; G16H 70/40

(52) U.S. Cl. CPC G06N 5/022; G16H 70/40

(57) ABSTRACT

A method and device for drug combination design by a high-throughput platform and machine learning for optimizing is provided, including: constructing an initial data set for machine learning using the high-throughput platform; inputting the initial data set into a plurality of machine learning models, and training the plurality of regression models; predicting unknown D-amino acid mixtures using the machine learning models and an efficient global optimization algorithm; and conducting experimental iterative feedback on a candidate mixture formula, and conducting high-throughput performance screening on drug combinations of D-amino acid mixtures and a plurality of antibiotics optimized by machine learning. The performance screened is the drug resistance of bacteria to antibiotics, and antibacterial efficiency and cytotoxicity of the drug combinations. The technical solution significantly improves the identification scale, efficiency, and repeatability of the drug combinations, and designs a low-toxicity and high-efficiency treatment scheme to solve the problem of bacterial infection.

### CROSS REFERENCE TO RELATED APPLICATION

This patent application claims the benefit and priority of Chinese Patent Application No. 202210845111.7 filed with the China National Intellectual Property Administration on Jul. 19, 2022, the disclosure of which is incorporated by reference herein in its entirety as part of the present application.

### TECHNICAL FIELD

The present disclosure relates to the technical field of drug combination design, and in particular, to a method and device for drug combination design by a high-throughput platform and machine learning for optimizing.

### BACKGROUND

Bacterial infection is the most important cause of implant failure, medical equipment damage, and even patient death. Constructing surfaces with bactericidal or antibacterial adhesion functions is the most common means to solve bacterial infection. However, once the wily bacteria escape the antibacterial agent and successfully attach to the surface, they can quickly form a biofilm, greatly weakening the effect of the bactericidal substance and causing serious infection. Therefore, the stubbornness of the biofilms makes them difficult to treat and eradicate, and the ideas of designing the traditional antibacterial coating cannot effectively combat the biofilms. Multiple antibacterial surfaces that can directly inhibit the formation of biofilms or eradicate pre-existing biofilms can effectively combat the biofilms, which ensures the long-term efficacy of bactericidal substances and reduces the risk of secondary infection in patients. In addition, it can also reduce the minimum inhibitory concentration of the bactericidal substances and enhance the biocompatibility of antibacterial surfaces.

D-amino acid has been proved to be an anti-biofilm drug with excellent biocompatibility. Studies have shown that the combined use of multiple D-amino acids has a more significant inhibitory effect on cell membrane formation than the use of a single D-amino acid. However, the traditional methods based on trial-and-error experiments are limited by the large number of samples, making it difficult to optimize the anti-biological performance of D-amino acid mixtures. How to quickly discover target D-amino acid mixtures with excellent properties is a huge challenge. Machine learning is an active learning method that starts with “a small dataset” and dynamically adds experimental results to the training data to speed up finding solutions to potential targets. But in regression studies, only the results of the model are used, and the lack of sampling points in the search space with the greatest uncertainty tends to limit the predicted value to the local minimum value. The key to the regression study is the acquisition function navigating the optimal solution in the latent space.

In addition, the drug combination of D-amino acids and antibiotics has a potential synergistic effect. The researchers claim that the introduction of D-amino acids can effectively improve the antibacterial effect of antibiotics and significantly reduce the cytotoxicity of antibiotics. How to quickly, accurately, and repeatably screen drug combinations to release the anti-biofilm potential of D-amino acids and endow antibiotics with broader application prospects is the current challenge.

### SUMMARY

In view of the problems in the prior art that the lack of sampling points in the search space with the greatest uncertainty tends to limit the predicted value to the local minimum value, and how to screen drug combinations quickly, accurately and repeatably, the present disclosure provides a method and device for drug combination design by a high-throughput platform and machine learning for optimizing.

To solve the above technical problem, the present disclosure provides the following technical solutions.

In an aspect, a method for drug combination design by a high-throughput platform and machine learning for optimizing is provided, which is applied to an electronic device and includes the following steps:

S1: constructing an initial training data set for machine learning, training and optimizing a plurality of preset machine learning regression models through the initial training data set, and selecting an optimal model;

S2: predicting, based on the optimal model, anti-biofilm performance of candidate mixtures through an efficient global optimization (EGO) algorithm to obtain a predicted performance value and an expected improvement (EI) value of each of the candidate mixtures;

S3: optimizing each of the candidate mixtures with the EI value as a standard to obtain a mixture ratio with excellent target performance, so as to obtain an optimized candidate mixture; and

S4: conducting drug combination on the optimized candidate mixture and a plurality of antibiotics, and conducting high-throughput performance screening on obtained drug combinations to screen out a low-toxicity and high-efficiency drug combination, so as to complete drug combination design by the high-throughput platform and machine learning for optimizing.

Optionally, constructing the initial training data set for machine learning, training and optimizing the plurality of preset machine learning regression models through the initial training data set, and selecting the optimal model in step S1 includes:

S11: characterizing a plurality of D-amino acids with anti-biofilm performance by crystal violet staining, and screening out top five D-amino acids in characterized performance results;

S12: combining the five D-amino acids in different ratios to form D-amino acid mixtures through the high-throughput platform, and characterizing anti-biofilm performance of the D-amino acid mixtures to construct and normalize the initial training data set, where the D-amino acid mixtures with different ratios are defined as the candidate mixtures;

S13: training the plurality of machine learning regression models through the initial training data set to obtain a mean square error of each of the machine learning regression models; and

S14: tuning hyperparameters of each of the machine learning regression models by a 10-fold cross-validation method, and selecting a machine learning regression model with a minimum mean square error as the optimal model.

Optionally, the initial training data set includes: an input data set and an output data set. The input data set comprises a compounding ratio of individual units in each candidate mixture, and the output data set comprises the anti-biofilm performance of each candidate mixture.

Optionally, step S2 further includes:

predicting each of the candidate mixtures n times by statistical inference, where n≥1,000, and a mean value of the predicted values is taken as the predicted performance value.

Optionally, optimizing each of the candidate mixtures with the EI value as the standard to obtain the mixture ratio with excellent target performance, so as to obtain the optimized candidate mixture includes:

S31: selecting a drug combination of a candidate mixture with a maximum EI value as a candidate formula of an experimental iteration, and obtaining a true value of the candidate formula through experiments;

S32: adding the true value of the candidate formula to the initial training data set, so as to expand the initial training data set; and

S33: repeating steps S2 to S32 on the expanded initial data set until the candidate formula meets preset requirements to obtain the mixture ratio with excellent target performance, so as to obtain the optimized candidate mixture.

Optionally, the preset requirements include that the experimental true value of the D-amino acid mixture is lower than all values in the initial training data set.

Optionally, conducting drug combination on the optimized candidate mixture and the plurality of antibiotics, and conducting high-throughput performance screening on obtained drug combinations to screen out the low-toxicity and high-efficiency drug combination, so as to complete drug combination design by the high-throughput platform and machine learning for optimizing in step S4 includes:

S41: screening, by the high throughput platform, the drug combinations using the plurality of antibiotics at different concentrations according to drug resistance of bacteria, to obtain screened drug combinations having the optimized candidate mixture and one or more of the plurality of antibiotics; and

S42: screening the screened drug combinations in terms of antibacterial performance and cytotoxicity using the high-throughput platform to obtain the low-toxicity and high-efficiency drug combination, so as to complete the drug combination design by the high-throughput platform and machine learning for optimizing.

Optionally, the low-toxicity and high-efficiency drug combination refers to a drug combination that has antibacterial efficiency greater than 90% and cell viability greater than 95% within 24 hours.

In an aspect, a device for drug combination design by a high-throughput platform and machine learning for optimizing is provided, which is applied to an electronic device and includes:

a model training module configured to construct an initial training data set for machine learning, train and optimize a plurality of preset machine learning regression models through the initial training data set, and select an optimal model;

a performance prediction module configured to predict anti-biofilm performance of candidate mixtures through an EGO algorithm based on the optimal model to obtain a predicted performance value and an EI value of each of the candidate mixtures;

a ratio optimization module configured to optimize each of the candidate mixtures with the EI value as a standard to obtain a mixture ratio with excellent target performance, so as to obtain an optimized candidate mixture; and

a drug combination module configured to conduct drug combination on the optimized candidate mixture and antibiotics, and conduct high-throughput performance screening on obtained drug combinations to screen out a low-toxicity and high-efficiency drug combination, so as to complete drug combination design by the high-throughput platform and machine learning for optimizing.

Optionally, the model training module is configured to conduct the following operations: characterizing a plurality of D-amino acids with anti-biofilm performance by crystal violet staining, and screening out top five D-amino acids in characterized performance results;

combining the five D-amino acids in different ratios to form D-amino acid mixtures through the high-throughput platform, and characterizing anti-biofilm performance of the D-amino acid mixtures to construct and normalize the initial training data set, where the D-amino acid mixtures with different ratios are defined as the candidate mixtures;

training the plurality of machine learning regression models through the initial training data set to obtain a mean square error of each of the machine learning regression models; and

tuning hyperparameters of each of the machine learning regression models by a 10-fold cross-validation method, and selecting a machine learning regression model with a minimum mean square error as the optimal model.

In an aspect, an electronic device is provided. The electronic device includes a processor and a memory. At least one instruction is stored in the memory. The at least one instruction is loaded and executed by the processor to implement the above-mentioned method for drug combination design by the high-throughput platform and machine learning for optimizing.

In an aspect, a computer-readable storage medium is provided. At least one instruction is stored in the storage medium. The at least one instruction is loaded and executed by the processor to implement the above-mentioned method for drug combination design by the high-throughput platform and machine learning for optimizing.

The above technical solutions in the examples of the present disclosure at least achieve the following beneficial technical effects:

In the above solution, (1) the design method combining the high-throughput platform and the machine learning strategy constructed by the present disclosure can quickly and accurately create the original data set for machine learning. Moreover, through the Bayesian optimization algorithm, the optimal solution in the latent space is efficiently navigated, local extreme values are avoided, and an excellent D-amino acid compounding method is found under the premise of a small number of iterations.

(2) The feasibility of combined treatment of D-amino acid-antibiotic drug combinations is explored using the high-throughput platform. The combined behaviors (synergy/antagonism) of drug combinations are quickly discovered. The application prospects of drug combinations are comprehensively characterized. The development efficiency is significantly improved. The research and development costs are effectively reduced. Technical guidance is provided for scientific research and application. New ideas are provided for the development of a low-toxicity and high-efficiency drug combination.

### BRIEF DESCRIPTION OF THE DRAWINGS

In order to describe the technical solutions in the examples of the present disclosure more clearly, the accompanying drawings required to describe the examples are briefly described below. Apparently, the accompanying drawings described below are only some examples of the present disclosure. Those of ordinary skill in the art may further obtain other accompanying drawings based on these accompanying drawings without inventive effort.

FIG. 1 is a flow chart of a method for drug combination design by a high-throughput platform and machine learning for optimizing according to one embodiment of the present disclosure;

FIG. 2 is a flow chart of a method for drug combination design by a high-throughput platform and machine learning for optimizing according to one embodiment of the present disclosure;

FIG. 3 is a fitting curve diagram of predicted values and experimental values of a machine learning model for an OD570 value of anti-biofilm performance of a method for drug combination design by a high-throughput platform and machine learning for optimizing according to one embodiment of the present disclosure;

FIG. 4 is a result diagram of experimental iterative feedback based on a Bayesian optimization algorithm of a method for drug combination design by a high-throughput platform and machine learning for optimizing according to one embodiment of the present disclosure;

FIG. 5 is a heat map of combined behaviors of D-amino acid mixtures and antibiotics of a method for drug combination design by a high-throughput platform and machine learning for optimizing according to one embodiment of the present disclosure;

FIG. 6A is a 24-hour cytotoxicity screening result diagram of a method for drug combination design by a high-throughput platform and machine learning for optimizing according to one embodiment of the present disclosure;

FIG. 6B is a 48-hour cytotoxicity screening result diagram of a method for drug combination design by a high-throughput platform and machine learning for optimizing according to one embodiment of the present disclosure;

FIG. 7A is a graph showing a growth inhibition efficiency of Pseudomonas aeruginosa by a method for drug combination design by a high-throughput platform and machine learning for optimizing according to one embodiment of the present disclosure;

FIG. 7B is a graph showing an anti-biofilm efficiency of Pseudomonas aeruginosa by a method for drug combination design by a high-throughput platform and machine learning for optimizing according to one embodiment of the present disclosure;

FIG. 8A is a schematic diagram of distribution of surface viable bacteria characterized by fluorescent confocal microscopy of a method for drug combination design by a high-throughput platform and machine learning for optimizing according to one embodiment of the present disclosure;

FIG. 8B is a schematic diagram of distribution of dead bacteria of a method for drug combination design by a high-throughput platform and machine learning for optimizing according to one embodiment of the present disclosure;

FIG. 9 is a block diagram of a device for drug combination design by a high-throughput platform and machine learning for optimizing according to one embodiment of the present disclosure; and

FIG. 10 is a schematic structural diagram of an electronic device according to one embodiment of the present disclosure.

### DETAILED DESCRIPTION OF THE EMBODIMENTS

To make the to-be-solved technical problems, technical solutions, and advantages of the present disclosure clearer, the present disclosure will be described in detail below with reference to the accompanying drawings and specific examples.

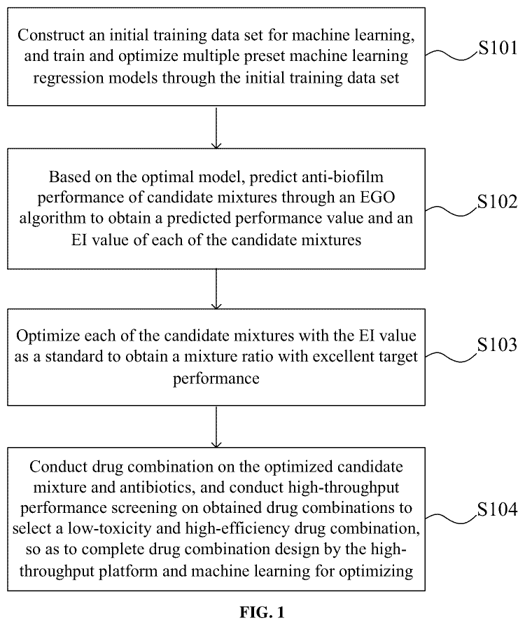

One embodiment of the present disclosure provides a method for drug combination design by a high-throughput platform and machine learning for optimizing. The method can be implemented by an electronic device, and the electronic device can be a terminal or a server. FIG. 1 is a flow chart of a method for drug combination design by a high-throughput platform and machine learning for optimizing, and the method can include:

S101: constructing an initial training data set for machine learning, training and optimizing a plurality of preset machine learning regression models through the initial training data set, and selecting an optimal model;

S102: predicting, based on the optimal model, anti-biofilm performance of candidate mixtures through an EGO algorithm to obtain a predicted performance value and an EI value of each of the candidate mixtures;

S103: optimizing each of the candidate mixtures with the EI value as a standard to obtain a mixture ratio with excellent target performance, so as to obtain an optimized candidate mixture; and

S104: conducting drug combination on the optimized candidate mixture and a plurality of antibiotics, and conducting high-throughput performance screening on the obtained drug combinations to screen out a low-toxicity and high-efficiency drug combination, so as to finish drug combination design by the high-throughput platform and machine learning for optimizing.

Optionally, step S101 of constructing the initial training data set for machine learning, training and optimizing the plurality of preset machine learning regression models through the initial training data set, and selecting the optimal model includes: S111: characterizing a plurality of D-amino acids with anti-biofilm performance by crystal violet staining, and screening out top five D-amino acids in characterized performance results;

S112: combining the five D-amino acids in different ratios to form D-amino acid mixtures through the high-throughput platform, and characterizing anti-biofilm performance of the D-amino acid mixtures to construct and normalize the initial training data set, where the D-amino acid mixtures with different ratios are defined as the candidate mixtures;

S113: respectively training the plurality of machine learning regression models using the initial training data set to obtain a mean square error of each of the machine learning regression models; and

S114: tuning hyperparameters of each of the machine learning regression models by a 10-fold cross-validation method, and selecting a machine learning regression model with a minimum mean square error as the optimal model.

Optionally, the initial training data set includes: an input data set and an output data set. The input data set includes a ratio of individual units in each candidate mixture, and the output data set includes the anti-biofilm performance of each candidate mixture.

Optionally, step S102 further includes: predicting each of the candidate mixtures n times by statistical inference, where n≥1,000, and a mean value of the predicted values is taken as the predicted performance value.

Optionally, step S103 of optimizing each of the candidate mixtures with the EI value as the standard to obtain the mixture ratio with excellent target performance, so as to obtain the optimized candidate mixture, includes:

S131: selecting a drug combination of a candidate mixture with a maximum EI value as a candidate formula of an experimental iteration, and obtaining a true value of the candidate formula through experiments.

S132: adding the true value of the candidate formula to the initial training data set, so as to expand the initial training data set; and

S133: repeating steps S102 to S132 on the expanded initial data set until the candidate formula meets preset requirements to obtain the mixture ratio with excellent target performance, so as to obtain the optimized candidate mixture.

Optionally, the preset requirements include that the experimental true value of the D-amino acid mixture is lower than all values in the initial training data set.

Optionally, step S104 of conducting drug combination on the optimized candidate mixture and the plurality of antibiotics, and conducting high-throughput performance screening on the obtained drug combinations to screen out the low-toxicity and high-efficiency drug combination, so as to finish the drug combination design by the high-throughput platform and machine learning for optimizing, includes:

S141: screening the drug combinations using the plurality of antibiotics at different concentrations according to drug resistance of bacteria through the high-throughput platform to obtain drug combinations having the optimized candidate mixture and one or more of the plurality of antibiotics; and

S142: screening the screened drug combinations in terms of antibacterial performance and cytotoxicity using the high-throughput platform to obtain the low-toxicity and high-efficiency drug combination, so as to finish the drug combination design by the high-throughput platform and machine learning for optimizing.

Optionally, the low-toxicity and high-efficiency drug combination refers to a drug combination that has antibacterial efficiency greater than 90% and cell viability greater than 95% within 24 hours.

In the embodiment of the present disclosure, an innovative method combining the high-throughput technology and the machine learning algorithm is provided to quickly and systematically identify drug combinations for treating microbial infections. In the case of insufficient data in prior art, the original data set is created using the high-throughput platform, and a complex relationship between a drug compounding ratio and anti-biofilm performance is unlocked through the machine learning model and the Bayesian optimization algorithm. In addition, the high-throughput platform is used to efficiently and cost-effectively screen drug combinations on multiple properties. The present disclosure provides a new method for designing high-efficiency and low-toxicity drug combinations against bacterial infection, which significantly improves the efficiency of drug combination design, and reduces the development cost.

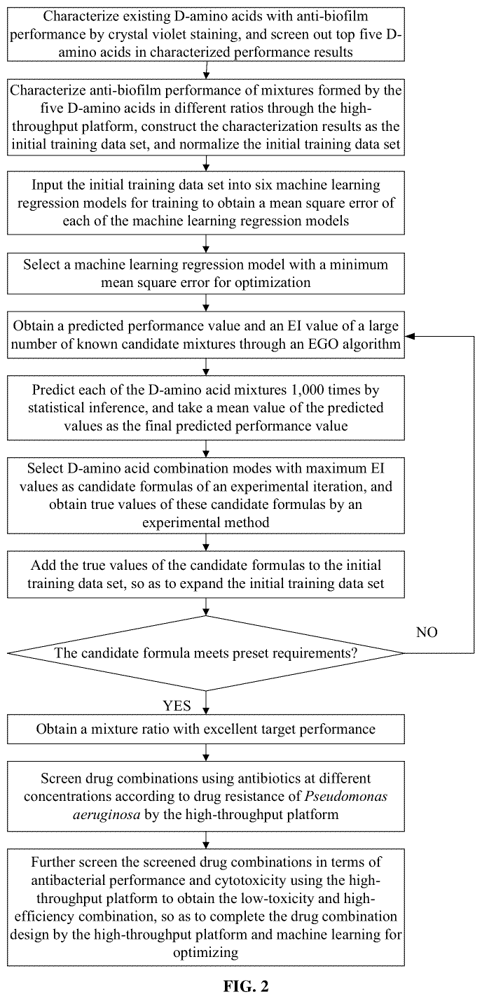

One embodiment of the present disclosure provides a method for drug combination design by a high-throughput platform and machine learning for optimizing. The method can be implemented by an electronic device, and the electronic device can be a terminal or a server. FIG. 2 is a flow chart of the method for drug combination design by the high-throughput platform and machine learning for optimizing, and a process of the method can include steps S201-S210.

S201: A plurality of D-amino acids with anti-biofilm performance are characterized by crystal violet staining, and the top five D-amino acids in characterized performance results are screened out.

In a feasible implementation, ten reported D-amino acids with anti-biofilm performance are characterized by crystal violet staining, and five of them with more excellent performance are selected.

S202: The five D-amino acids are formed into D-amino acid mixtures in different ratios through the high-throughput platform and anti-biofilm performance of the D-amino acid mixtures is characterized to construct and normalize the initial training data set. The D-amino acid mixtures with different ratios are defined as the candidate mixtures.

In a feasible implementation, the main device of the high-throughput platform is a multi-functional non-contact microarray printer that can accurately pipette liquid.

In a feasible implementation, the compounding method of the D-amino acid mixture may be monovalent, binary, ternary, quaternary, or pentavalent.

S203: The plurality of machine learning regression models are trained through the initial training data set to obtain a mean square error of each of the machine learning regression models.

S204: Hyperparameters of each of the machine learning regression models are tuned by a cross-validation method, and a machine learning regression model with a minimum mean square error is selected as the optimal model.

In a feasible implementation, the initial training data set includes: an input data set and an output data set. The input data set includes a compounding ratio of units in each D-amino acid mixture, and the output data set includes the anti-biofilm performance of the mixtures.

In a feasible implementation, a ratio of the training set to the test set for training the model is set to 4:1.

S205: Based on the optimal model, anti-biofilm performance of each candidate mixture is predicted through an EGO algorithm to obtain a predicted performance value and an EI value of each of the candidate mixtures.

In a feasible implementation, a process of obtaining the predicted performance value of each of the candidate mixtures includes: each of the candidate mixtures is predicted n times by statistical inference, where n≥1,000, and a mean value of the predicted values is taken as the predicted performance value.

S206: A drug combination of a candidate mixture with a maximum EI value is selected as a candidate formula of an experimental iteration, and a true value of the candidate formula is obtained through experiments.

S207: The true value of the candidate formula is added to the initial training data set, so as to expand the initial training data set.

S208: Steps S205 to S207 are repeated on the expanded initial data set until the candidate formula meets preset requirements to obtain the mixture ratio with excellent target performance, so as to obtain the optimized candidate mixture.

In a feasible implementation, the preset requirements include that the experimental true value of the D-amino acid mixtures is lower than all values in the initial training data set, and the change trend of the true value is gradually flattened, that is, the difference between a true value and a preceding true value is reduced within 10%.

In the embodiments of the present disclosure, a series of compounding methods of D-amino acid mixtures are explored and found, and the compounding methods of mixtures whose anti-biofilm performance is superior to those of all current literature reports are successfully found.

S209: Drug resistance of bacteria is screened with a plurality of antibiotics at different concentrations through the high-throughput platform to obtain drug combinations having the optimized candidate mixtures and the antibiotics.

S210: The drug combinations in terms of antibacterial performance and cytotoxicity are screened using the high-throughput platform to screen out the low-toxicity and high-efficiency drug combination, so as to complete the drug combination design by the high-throughput platform and machine learning for optimizing.

In a feasible implementation, a low-toxicity and high-efficiency standard is: within 24 hours, antibacterial efficiency is greater than 90%, and cell viability is greater than 95%.

In a feasible implementation, the drug combination refers to a D-amino acid mixture and antibiotics. Drug resistance screening makes use of the concentration of antibiotics as a reference for processing subsequent drug combinations. For example, if the bacteria have strong drug resistance, the concentration of antibiotics in the drug combinations shall be increased accordingly to ensure the bactericidal effect. That is, the aqueous solution of the drug combination directly acts on the bacteria, for example, gram-positive bacteria such as Streptococcus and Staphylococcus aureus and gram-negative bacteria such as Escherichia coli and Pseudomonas aeruginosa.

The solution of the present disclosure is described in detail below through four groups of experimental data.

### Embodiment 1

a. The published literature was sorted out, and ten D-amino acids with anti-biofilm performance were selected. A high-throughput platform was used to quickly evaluate the anti-biofilm performance of these ten D-amino acids at a concentration of 100 μm against Pseudomonas aeruginosa, five of which with more excellent performance were screened out, and were subjected to monovalent, binary, ternary, quaternary, and pentavalent mixing. Ten rounds of high-throughput characterization of the anti-biological performance of the mixture at a final concentration of 100 μm resulted in approximately 1,000 pieces of data.

In one embodiment of the present disclosure, the main frame of the high-throughput platform was a multi-functional non-contact microarray printer, which was composed of modules such as a multi-functional workbench, a liquid suction porous plate, a piezoelectric pipetting needle, a real-time camera, and a cleaning/drying device. The working mode according to the preset program could summarily include the steps of liquid suction, optimization, pipetting, verification, washing, and drying. The piezoelectric pipetting needle of the microarray printer could precisely dispense volumes as low as picoliters to various well plates and material surfaces, and provide real-time images of pipetting. The real-time camera could optimize the pipetting parameters and verify the working state of the needle to ensure accuracy and repeatability of pipetting.

b. All data was normalized to remove obvious offset values, and the original data set was constructed with the average value of the remaining data.

c. A compounding method→anti-biological performance regression model was established. A mixture ratio in the original data set was taken as the input and the anti-biological performance was taken as the output. The plurality of regression models were trained. Hyperparameters of each model were tuned by 10-fold cross-validation. A ratio of the training set to a test set for training the model was 4:1. The training set was used for the training of the regression model, the test set was used to test accuracy of the regression model, and the mean square error was used to evaluate accuracy of the regression model. In practical application, the multiples of cross-validation, the means of hyperparameter optimization, and the criteria of model accuracy could be adjusted according to specific situations.

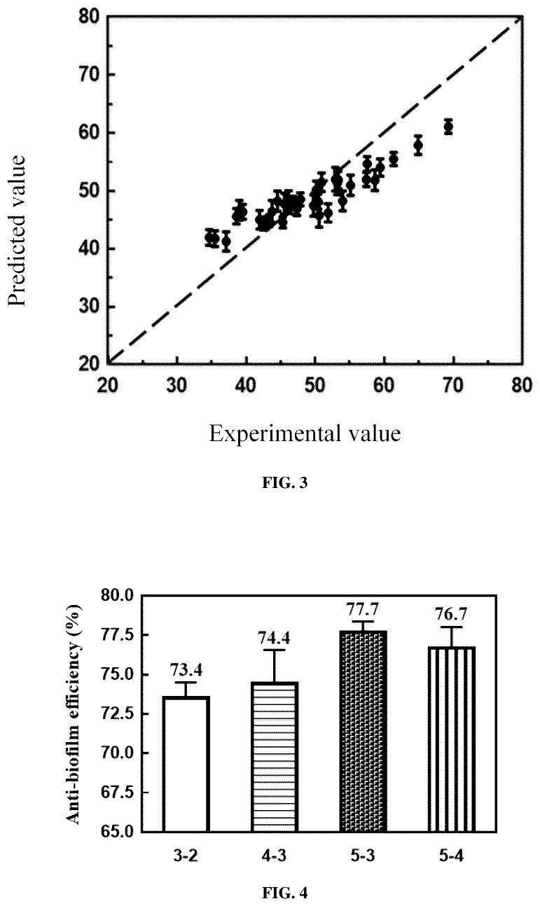

A scatter plot was drawn according to the experimental true values and the predicted values of the model obtained from the above steps. As shown in FIG. 3, the specific method was as follows: the scatter plot was drawn by taking the experimental data obtained by the high-throughput platform as the abscissa and the predicted value of the machine learning model as the ordinate. When the scatter point was closer to the 45° line, it meant that the experimental value and the predicted value were closer, accuracy of the model was more excellent, accuracy of the random forest model was more excellent, and the mean square error was 46.24.

d. The optimal model in step c was selected. The predicted performance values and the EI values of a large number of D-amino acid mixtures were obtained by the optimal model combining statistical inference and the Bayesian optimization algorithm. The data with the maximum EI value was selected as the iterative candidate. The proportion of all D-amino acids was between 0 and 100%, the stride was set to 5%, and there were 10,626 pieces of predicted data. The crystal violet staining was used to evaluate the anti-biological properties of the compounding method, and the results were added to the original data set. This process was repeated until a D-amino acid mixture with excellent target performance was found and no significant change occurred in this performance in the iterative process.

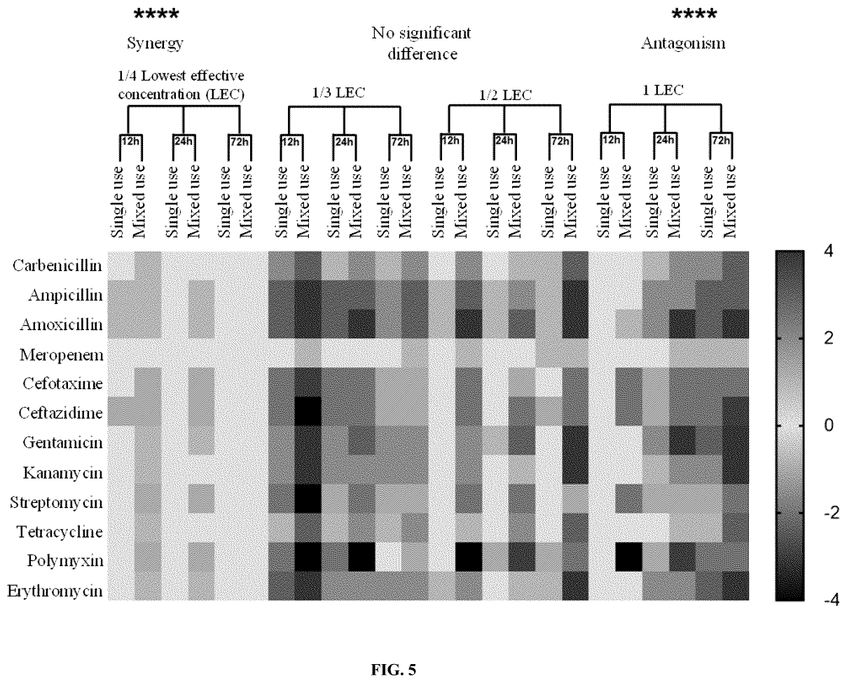

Experimental iterations were conducted according to the method in step d, and there were a total of four amino acid mixtures having more excellent performance than all samples in the original data set, as shown in FIG. 4. The compounding methods of the mixtures are shown in Table 1. Finally, in cycle 5-3, the optimal mixture was successfully found, which consisted of 15% D-tyrosine, 15% D-tryptophan, 60% D-leucine, 10% D-phenylalanine, and 0% D-proline, and had optimal anti-biofilm efficiency reported so far.

TABLE 1
Prediction result table of D-amio acid mixtures

| | D-amino acid | Anti-biofilm | |
|---|---|---|---|
| Cycle number | compounding method | efficiency | EI |
| 3-2 | 10Tyr-5Trp-70Leu-15Phe-0Pro | 73.4618 | 0.1226 |
| 4-3 | 5Tyr-25Trp-50Leu-20Phe-0Pro | 74.4453 | 0.0618 |
| 5-3 | 15Tyr-15Trp-60Leu-10Phe-0Pro | 77.6918 | 0.0190 |
| 5-4 | 15Tyr-15Trp-55Leu-10Phe-5Pro | 76.6935 | 0.0378 |

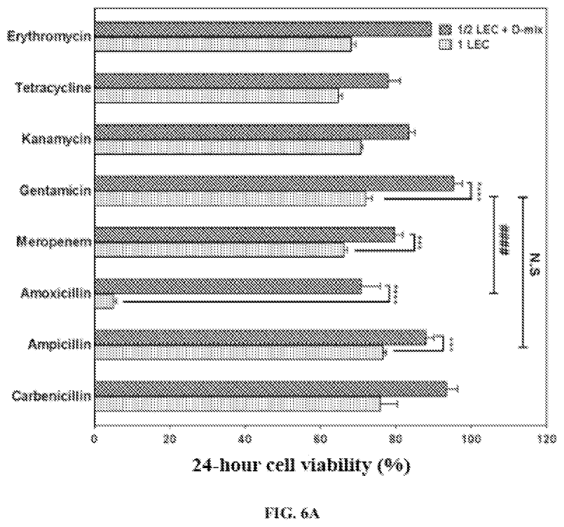

e. High-throughput screening was conducted on the feasibility of combined treatment of 288 D-amino acid mixture-antibiotic drug combinations optimized by machine learning, and the screening results were presented in the form of a heat map, as shown in FIG. 5. In the screening of 12 antibiotics, four types of antibiotics, including 0-lactamases, aminoglycosides, tetracyclines, and macrolides, and D-amino acid mixtures were synergistic with each other, and had the potential for combined treatment.

### Embodiment 2

The difference between Embodiment 2 and Embodiment 1 was as follows: in step a, the final concentration of D-amino acid and its mixture could also be set to 500 μm; in step c, Gaussian regression had the optimal model accuracy, and the mean square error was 42.62; and in step d, the optimal mixture consisted of 15% D-tyrosine, 15% D-tryptophan, 55% D-leucine, 10% D-phenylalanine, and 5% D-proline, and had the optimal anti-biofilm efficiency reported so far.

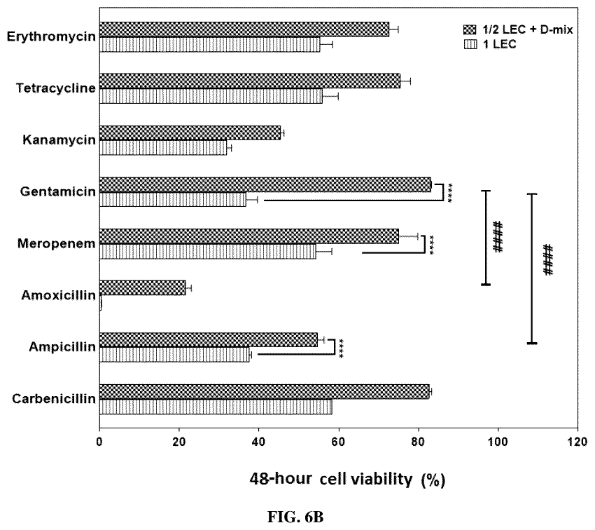

High-throughput screening was conducted on the cytotoxicity of the D-amino acid mixture-antibiotic drug combination optimized by machine learning, as shown in FIG. 6A and FIG. 6B. In the screening of 8 antibiotics, D-amino acid mixture-gentamicin had the lowest cytotoxicity and had the potential for combined treatment.

### Embodiment 3

The difference was as follows: in step a, the final concentration of D-amino acid and its mixture could also be set to 200 μm; in step c, Gaussian regression had the optimal model accuracy, and the mean square error was 45.25; and in step d, the stride was set to 2%, and there were 316,251 pieces of predicted data.

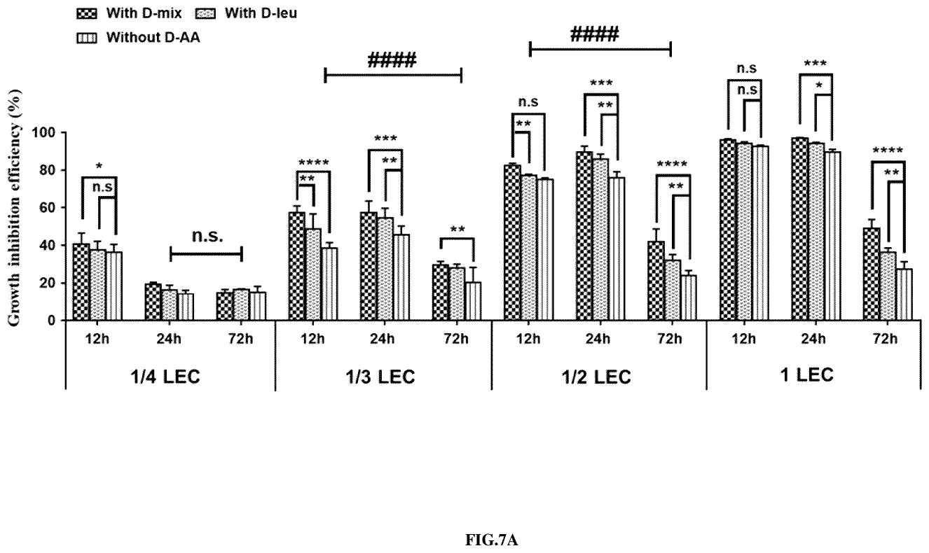

High-throughput screening was conducted on the growth inhibition efficiency of the D-amino acid mixture-antibiotic drug combination optimized by machine learning on Pseudomonas aeruginosa and its biofilm, and the most promising D-amino acid mixture-gentamicin drug combination was comprehensively characterized, as shown in FIGS. 7A-7B. The combination of 200 μm of D-amino acid mixture and 4 mg/l of gentamicin could kill at least 90% of Pseudomonas aeruginosa within 24 h, and could inhibit 96% of biofilm formation. The D-amino acid mixture optimized by machine learning released the anti-biofilm potential of D-amino acids, providing new therapeutic possibilities for the low-toxicity and high-efficiency drug combination.

### Embodiment 4

The difference was as follows: in step a, the anti-biofilm performance of these ten D-amino acids at a concentration of 200 μm against Staphylococcus aureus was quickly evaluated, six of which with more excellent performance were screened out and were subjected to monovalent, binary, ternary, quaternary, pentavalent, and hexavalent mixing. Ten rounds of high-throughput characterization of the anti-biological performance of the mixture at a final concentration of 200 μm resulted in approximately 1,500 pieces of data; in step c, Gaussian regression had the optimal model accuracy, and the mean square error was 46.98; and in step d, the stride was set to 10%, and there were 2,082 pieces of predicted data.

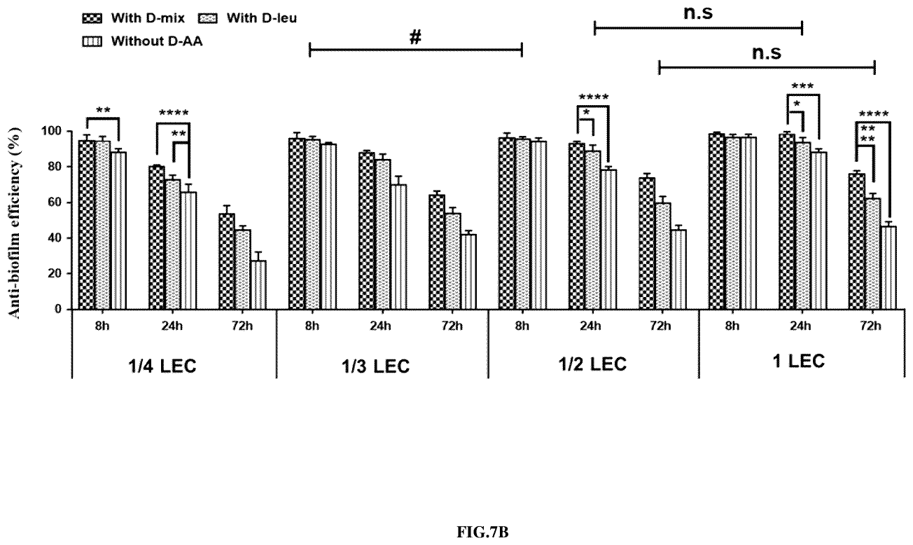

The antibacterial performance of the D-amino acid mixture-carbenicillin drug combination optimized by machine learning against Staphylococcus aureus was evaluated, and the evaluation results were presented by fluorescent confocal live-dead staining, as shown in FIG. 8A and FIG. 8B. When the surface was exposed to the drug combination, the bacteria were highly dispersed due to the lack of an organized biofilm structure, and the number of viable bacteria decreased dramatically and the number of dead bacteria increased significantly in the field of view. This was due to the introduction of the D-amino acid mixture, which greatly inhibited the formation of biofilms. In the absence of biofilm protection, the resistance of bacteria to antibiotics was greatly reduced, and a small amount of antibiotics could cause excellent killing ability to bacteria. The D-amino acid mixture-carbenicillin drug combination greatly reduced the dosage requirements of carbenicillin without sacrificing its excellent antibacterial effect, solved the cytotoxicity problem of carbenicillin, and significantly inhibited the growth of Staphylococcus aureus and the formation of biofilm.

In the embodiment of the present disclosure, D-amino acid is an anti-biofilm drug with excellent application prospects, but its development is limited by the difficulty in finding the optimal compounding ratio of the D-amino acid mixture to release its real application potential. The traditional trial-and-error method is extremely expensive and may not characterize tens of thousands of compounding methods in a short time. The design method combining the high-throughput platform and the machine learning strategy constructed by the present disclosure may quickly and accurately create the original data set for machine learning. Moreover, through the Bayesian optimization algorithm, the optimal solution in the latent space is efficiently navigated, local extreme values are avoided, and an excellent D-amino acid compounding method is found under the premise of a small number of iterations.

The high-throughput platform is used to explore the feasibility of combined treatment of D-amino acid-antibiotic drug combinations, quickly discover the combined behaviors (synergy/antagonism) of drug combinations, and comprehensively characterize the application prospects of drug combinations, which significantly improves the development efficiency, effectively reduces the research and development costs, and provides technical guidance for scientific research and application and a new idea for the development of the low-toxicity and high-efficiency drug combination.

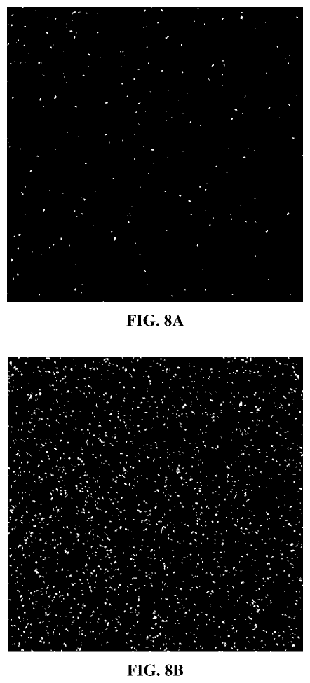

FIG. 9 is a block diagram of a device for drug combination design by a high-throughput platform and machine learning for optimizing according to an exemplary embodiment. With reference to FIG. 9, the device 300 includes a model training module 310, a performance prediction module 320, a ratio optimization module 330, and a drug combination module 340.

The model training module 310 is configured to construct an initial training data set for machine learning through the high-throughput platform, and train and optimize preset machine learning regression models through the initial training data set.

The performance prediction module 320 is configured to predict anti-biofilm performance of candidate mixtures through an EGO algorithm based on the optimal model to obtain a predicted performance value and an EI value of each of the candidate mixtures.

The ratio optimization module 330 is configured to optimize each of the candidate mixtures with the EI value as a standard to obtain a mixture ratio with excellent target performance.

The drug combination module 340 is configured to conduct drug combination on the optimized candidate mixture and antibiotics, and conduct high-throughput performance screening on obtained drug combinations to obtain a low-toxicity and high-efficiency drug combination, so as to finish drug combination design by the high-throughput platform and machine learning for optimizing.

Optionally, the model training module 310 is configured to: characterize existing D-amino acids with anti-biofilm performance by crystal violet staining, and screen out top five D-amino acids on characterization performance;

characterize anti-biofilm performance of mixtures formed by the five D-amino acids in different ratios through the high-throughput platform, construct the characterization results as an initial training data set, and normalize the initial training data set;

input the initial training data set into six machine learning regression models for training to obtain a mean square error of each of the machine learning regression models; and

select a machine learning regression model with a minimum mean square error for optimization.

Optionally, the initial training data set includes: an input data set and an output data set. The input data set includes a ratio of individual units in each D-amino acid mixture, and the output data set includes the anti-biofilm performance of each mixture.

Optionally, the performance prediction module 320 is further configured to predict each of the D-amino acid mixtures 1,000 times by statistical inference, and a mean value of the predicted values is taken as the final predicted performance value.

Optionally, the ratio optimization module 330 is configured to: select D-amino acid combinations with a maximum EI value as candidate formulas of an experimental iteration, and obtain true values of these candidate formulas by an experimental method;

add the true values of the candidate formulas to the initial training data set, so as to expand the initial training data set; and

conduct repeatedly performance prediction and initial training data set expansion on the expanded initial data set until the candidate formula meets preset requirements to obtain a mixture ratio with excellent target performance.

Optionally, the preset requirements include that the experimental true value of the D-amino acid mixtures is lower than all values in the initial training data set, and the change trend of the true values is gradually flattened.

Optionally, the drug combination module 340 is configured to: screen drug resistance of Pseudomonas aeruginosa using the high-throughput platform, where there are 98 antibiotics of different concentrations used for screening;

screen antibacterial performance of the drug combinations using the high-throughput platform, where there are 288 drug combinations used for the screening; and

screen cytotoxicity of the drug combinations using the high-throughput platform, where there are 32 drug combinations used for the screening, and finally screen out a low-toxicity and high-efficiency drug combination, so as to complete the drug combination design by the high-throughput platform and machine learning for optimizing.

Optionally, a low-toxicity and high-efficiency standard is: within 24 hours, antibacterial efficiency is greater than 90%, and cell viability is greater than 95%.

In the embodiment of the present disclosure, an innovative method combining the high-throughput technology and the machine learning algorithm is provided to quickly and systematically identify drug combinations for treating microbial infections. In the case of insufficient data in prior art, an original data set is created using the high-throughput platform, and a complex relationship between a drug compounding ratio and anti-biofilm performance is unlocked through the machine learning model and the Bayesian optimization algorithm. In addition, the high-throughput platform is used to efficiently and cost-effectively screen drug combinations for multiple properties. The present disclosure provides a new method for designing a high-efficiency and low-toxicity drug combination against bacterial infection, which significantly improves the efficiency of drug combination design, and reduces the development cost.

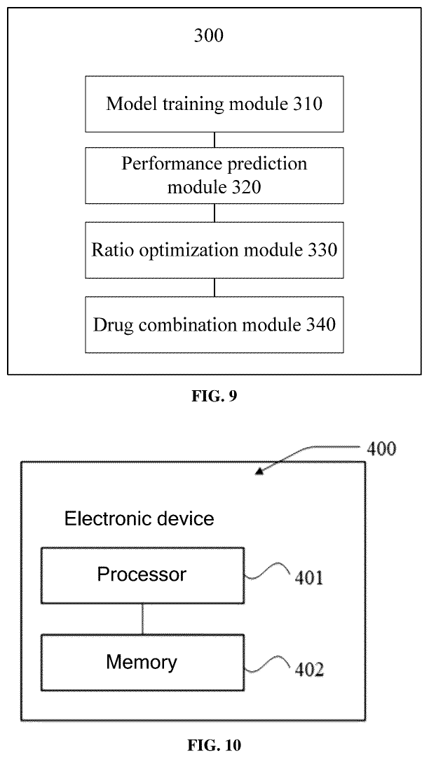

FIG. 10 is a schematic structural diagram of an electronic device 400 according to one embodiment of the present disclosure. The electronic device 400 may vary greatly due to different configurations or performances, and may include one or more processors (central processing units (CPUs)) 401 and one or more memories 402, where at least one instruction is stored in the memory 402. The at least one instruction is loaded and executed by the processor 401 to implement the steps of the below method for drug combination design by a high-throughput platform and machine learning for optimizing:

S1: constructing an initial training data set for machine learning, training and optimizing a plurality of preset machine learning regression models using the initial training data set; and selecting an optimal model;

S2: predicting, based on the optimal model, anti-biofilm performance of candidate mixtures by an EGO algorithm to obtain a predicted performance value and an EI value of each of the candidate mixtures;

S3: optimizing each of the candidate mixtures with the EI value as a standard to obtain a mixture ratio with excellent target performance, so as to obtain an optimized candidate mixture; and

S4: conducting drug combination on the optimized candidate mixture and antibiotics, and conducting high-throughput performance screening on obtained drug combinations to screen out a low-toxicity and high-efficiency drug combination, so as to complete drug combination design by the high-throughput platform and machine learning for optimizing.

In an exemplary embodiment, a computer-readable storage medium, such as a memory including instructions, is further provided, and the instructions can be executed by a processor in a terminal to complete the above-mentioned method for the drug combination design by a high-throughput platform and machine learning for optimizing. For example, the computer-readable storage medium may be a read-only memory (ROM), a random access memory (RAM), a compact disc read-only memory (CD-ROM), a magnetic tape, a floppy disk, an optical data storage device, or the like.

Those of ordinary skill in the art can understand that all or some of the steps in the foregoing examples may be implemented by hardware, or by instructing related hardware by using a program. The program may be stored in a computer-readable storage medium. The storage medium may be a read-only memory, a disk, a compact disc, or the like.

The foregoing descriptions are merely descriptions of preferred examples of the present disclosure, and are not intended to limit the present disclosure. Any modifications, equivalent replacements and improvements made within the spirit and principle of the present disclosure should fall into the scope of the present disclosure.

1. A method for drug combination design by a high-throughput platform and machine learning for optimizing, comprising:
S1: constructing an initial training data set for machine learning, training and optimizing a plurality of preset machine learning regression models through the initial training data set, and selecting an optimal model;
S2: predicting, based on the optimal model, anti-biofilm performance of candidate mixtures through an efficient global optimization (EGO) algorithm to obtain a predicted performance value and an expected improvement (D) value of each of the candidate mixtures;
S3: optimizing each of the candidate mixtures with the EI value as a standard to obtain a mixture ratio with excellent target performance, so as to obtain an optimized candidate mixture; and
S4: conducting drug combination on the optimized candidate mixture and a plurality of antibiotics, and conducting high-throughput performance screening on obtained drug combinations to screen out a low-toxicity and high-efficiency drug combination, so as to complete the drug combination design by the high-throughput platform and machine learning for optimizing.

2. The method according to claim 1, wherein constructing the initial training data set for machine learning, training and optimizing the plurality of preset machine learning regression models through the initial training data set, and selecting the optimal model in step S1 comprises:
S11: characterizing a plurality of D-amino acids with anti-biofilm performance by crystal violet staining, and screening out top five D-amino acids in characterized performance results;
S12: combining the five D-amino acids in different ratios to form D-amino acid mixtures through the high-throughput platform, and characterizing anti-biofilm performance of the D-amino acid mixtures to construct and normalize the initial training data set, wherein the D-amino acid mixtures with different ratios are defined as the candidate mixtures;
S13: training the plurality of machine learning regression models through the initial training data set to obtain a mean square error of each of the machine learning regression models; and
S14: tuning hyperparameters of each of the machine learning regression models by a 10-fold cross-validation method, and selecting a machine learning regression model with a minimum mean square error as the optimal model.

3. The method according to claim 2, wherein the initial training data set comprises: an input data set and an output data set, wherein the input data set comprises a compounding ratio of individual units in each candidate mixture, and the output data set comprises the anti-biofilm performance of each candidate mixture.

4. The method according to claim 2, wherein step S2 further comprises:
predicting each of the candidate mixtures n times by statistical inference, wherein n≥1,000, and a mean value of the predicted values is taken as the predicted performance value.

5. The method according to claim 2, wherein optimizing each of the candidate mixtures with the EI value as the standard to obtain the mixture ratio with excellent target performance, so as to obtain the optimized candidate mixture in step S3 comprises:
S31: selecting a drug combination of a candidate mixture with a maximum EI value as a candidate formula of an experimental iteration, and obtaining a true value of the candidate formula through experiments;
S32: adding the true value of the candidate formula to the initial training data set, so as to expand the initial training data set; and
S33: repeating steps S2 to S32 on the expanded initial data set until the candidate formula meets preset requirements to obtain the mixture ratio with excellent target performance, so as to obtain the optimized candidate mixture.

6. The method according to claim 5, wherein the preset requirements comprise that the experimental true value of the candidate mixture is lower than all values in the initial training data set.

7. The method according to claim 5, wherein conducting drug combination on the optimized candidate mixture and the plurality of antibiotics, and conducting high-throughput performance screening on obtained drug combinations to screen out the low-toxicity and high-efficiency drug combination, so as to complete the drug combination design by the high-throughput platform and machine learning for optimizing in step S4 comprises:
S41: screening, by the high throughput platform, the drug combinations using the plurality of antibiotics at different concentrations according to drug resistance of bacteria, to obtain screened drug combinations having the optimized candidate mixture and one or more of the plurality of antibiotics; and
S42: screening the screened drug combinations in terms of antibacterial performance and cytotoxicity using the high-throughput platform to obtain the low-toxicity and high-efficiency drug combination, so as to complete the drug combination design by the high-throughput platform and machine learning for optimizing.

8. The method according to claim 7, wherein in step S42, the low-toxicity and high-efficiency drug combination refers to a drug combination that has antibacterial efficiency greater than 90% and cell viability greater than 95% within 24 hours.

9. A device for drug combination design by a high-throughput platform and machine learning for optimizing, comprising:
a model training module, configured to construct an initial training data set for machine learning, train and optimize a plurality of preset machine learning regression models through the initial training data set, and select an optimal model;
a performance prediction module, configured to determine an algorithm in the optimal model, and predict anti-biofilm performance of candidate mixtures through the algorithm to obtain an expected improvement (EI) value of each of the candidate mixtures;
a ratio optimization module, configured to predict the anti-biofilm performance of the candidate mixtures through an efficient global optimization (EGO) algorithm based on the optimal model to obtain a predicted performance value and an EI value of each of the candidate mixtures; and
a drug combination module, configured to conduct drug combination on the optimized candidate mixture and antibiotics, and conduct high-throughput performance screening on obtained drug combinations to obtain a low-toxicity and high-efficiency drug combination, so as to complete the drug combination design by the high-throughput platform and machine learning for optimizing.

10. The device according to claim 9, wherein the model training module is configured to:
characterize a plurality of D-amino acids with anti-biofilm performance by crystal violet staining, and screen out top five D-amino acids in characterized performance results;
combine the five D-amino acids in different ratios to form D-amino acid mixtures through the high-throughput platform, and characterize anti-biofilm performance of the D-amino acid mixtures to construct and normalize the initial training data set, wherein the D-amino acid mixtures with different ratios are defined as the candidate mixtures;
train the plurality of machine learning regression models through the initial training data set to obtain a mean square error of each of the machine learning regression models; and
tune hyperparameters of each of the machine learning regression models by a cross-validation method, and select a machine learning regression model with a minimum mean square error as the optimal model.
